(19) United States
(12) Patent Application Publication
(10) Pub. No.: US 2019/0252059 A1
(43) Pub. Date: Aug. 15, 2019

(54) Real-Time Targeted Warning System

(71) Applicants: Brad Gleason, Erie, PA (US); Amy Danzer, Erie, PA (US)

(72) Inventors: Brad Gleason, Erie, PA (US); Amy Danzer, Erie, PA (US)

(21) Appl. No.: 16/271,597

(22) Filed: Feb. 8, 2019

Related U.S. Application Data
(60) Provisional application No. 62/628,738, filed on Feb. 9, 2018.

(51) Int. Cl. G16H 20/70; G06Q 50/00; G16H 40/67; G06N 5/04; H04L 29/08

(52) U.S. Cl. CPC G16H 20/70; G06Q 50/01; G16H 40/67; G01S 19/13; H04L 67/306; H04L 67/22; G06N 5/048

(57) ABSTRACT

What is presented is a computer-implemented method for behavioral prediction of mental health clients. A profile is created for each mental health client. The profile includes a framework for alerts to be generated. The framework comprises client specific deidentified data points, client specific weights assigned to each data point, and client specific triggering events based on each data point. Each mental health client is provided with a cell phone enabled with a GPS tracker and social media software applications. The created profile is associated with the cell phone and the social media software applications. The cell phone and the social media software applications are then monitored over time to identify data points and triggering events to generate a log of triggering events. The assigned weights are applied to each triggering event to determine whether or not an alert is generated. Historical patterns are analyzed to optimize the accuracy of future alerts.

### BACKGROUND

Individuals with mental health diagnoses for which they have sought professional metal health services often require as many tools as they can get to help them address their issues. From an individual's perspective, it is often very helpful to have a variety of aids. From the metal health service provider's perspective, there is a need to be able to monitor at-risk individuals and to divert them to resources and assistance when necessary. From society's perspective, there is a need to be able to monitor at-risk individuals and anticipate when intervention by appropriate entities is required.

### SUMMARY

What is presented is a computer-implemented method for behavioral prediction of mental health clients' escalation. A profile is created for each mental health client. The profile includes a framework for alerts to be generated. The framework comprises client specific deidentified data points, client specific weights assigned to each data point, and client specific triggering events based on each data point. Each mental health client is provided with a cell phone enabled with a GPS tracker and social media software applications. An agreement is obtained from the mental health client to accept and use the cell phone and allow access to the data generated by it.

The created profile is associated with the cell phone and the social media software applications. The cell phone and the social media software applications are then monitored over time to identify data points and triggering events to generate a log of triggering events. Time stamps and location data are recorded for the GPS tracker, postings from social media software applications, and case logs provided by social service agencies. In some instances, third parties are deidentified from these sources. Language analysis, sentiment analysis, and emotion analysis are applied to data points obtained from social media software applications and case logs provided by social service agencies. The assigned weights are applied to each triggering event to determine whether or not an alert is generated. Support or intervention personnel are contacted when an alert is generated. Historical patterns are analyzed to optimize the accuracy of future alerts.

The client specific data points include any or all of demographic, historical, clinical, diagnostic, support system information, recordable behaviors, schedules, prohibited GPS locations, prohibited GPS zones, permitted GPS locations, and diagnoses.

Those skilled in the art will realize that this invention is capable of embodiments that are different from those shown and that details of the devices and methods can be changed in various manners without departing from the scope of this invention. Accordingly, the drawings and descriptions are to be regarded as including such equivalent embodiments as do not depart from the spirit and scope of this invention.

### BRIEF DESCRIPTION OF DRAWINGS

For a more complete understanding and appreciation of this invention, and its many advantages, reference will be made to the following detailed description taken in conjunction with the accompanying drawings.

FIG. 1 shows a schematic that illustrates the implementation of the monitoring system described herein;

FIG. 2 shows a schematic that illustrates sentiment and emotional analysis of raw text; and

FIG. 3 is a block diagram illustrates an exemplary computing device.

### DETAILED DESCRIPTION

Referring to the drawings, variations of corresponding parts in form or function that are depicted in the figures are described. It will be understood that variations in the embodiments can generally be interchanged without deviating from the invention.

Behavioral prediction of mental health clients is an important issue that needs a solution. Recent mass casualty incidents in the United States have inevitably pointed to individuals with known mental health issues that might have been avoided with early intervention and diversion. What is presented is a system and a computer-implemented method for behavior health prediction of mental health clients to improve diversionary methods through new technology that identifies when early intervention is needed.

The system and method are voluntary and require the acquiescence and consent of the mental health patient. It works on the assumption that a person's movements and social interactions form a baseline from which predictions of behavior and warnings of potential negative behavior can be flagged for intervention by the appropriate parties. The system creates a dashboard though which mental health patients can be monitored for risky activity that would warrant intervention or diversion. The dashboard allows mental health workers to store and access case management logs and allows for a continuity of treatment between the slew of social services, medical and mental health providers, and emergency and support personnel that is currently not available.

A profile is created for each mental health client with a framework for alerts to be generated. The framework comprises client specific data points. These data points include but are not limited to demographic data, historical data, clinical data, diagnostic information, support system information, the recordable behaviors of the mental health client, their schedules for work, doctor's appointment, and other activities, prohibited GPS locations and zones, permitted GPS locations, and medical and mental health diagnoses. Some of the locations that the mental health client may be prohibited from could be whole classes of locations for which specific data may not be available. For example, if the mental health client is prohibited for approaching or frequenting schools, day care centers, gun shops, or liquor stores, while some of these locations may be known, it may not be apparent when new locations open or old ones close. In such instances, classes of locations could be specified and cross-linked with publicly available information from such services as Google Maps, etc. Some or all this information could be deidentified to protect the privacy of third parties.

The relevancy of each data point can vary significantly from person to person. For example, a mental health client spending significant amount of time at a bar could be an issue if that person is an alcoholic but may mean less if that person works there. Similarly, a mental health client who makes an unscheduled stop at a gun store for a significant amount of time would carry more weight, but if that person happens to be stopping at a traffic light that is adjacent a gun store, that should not raise a warning. Similarly, there may be third parties that the mental health client should be avoiding and there should be heightened awareness if a mental health client is close to or frequenting those locations where those third parties work or live. Many of these events or limitations are situations that are specific to the particular mental health client, therefore, client specific weights are assigned for each data point in the profile.

There are certain instances that are absolute triggers for warnings. For example, a mental health client with a history of violence attending a gun show should raise significant warnings. In addition, while there are events or activities that by themselves in isolation or occasionally do not mean much, it could be that the detection of specific data points over time could indicate the presence of a problem. In those instances, the combination of weights assigned to each data point could accumulate to a point that intervention is called for or at least an investigation of the noted pattern. In either case, client specific triggering events are created in the profile for each data point. Specific classes of locations could be combined with a warning radius if the mental health client spends too much time at specific locations or crosses within a specified radius of specific locations.

All these data points are location specific and there are several ways to track a person's location including wrist and ankle trackers that are used for monitoring parolees and criminals. However, these tools leave much to be desired in the treatment of mental health clients where maintaining a sense of normally is a priority. Therefore, under this system, each mental health client is provided with a cell phone that is enabled with a GPS tracker. The mental health client is asked to always keep the cell phone with them and to use it as their exclusive communications device. Part of the treatment protocol will include obtaining agreements by the mental health client to accept and use the cell phone and to allow access to the data generated by it.

The cell phone provided would be preloaded with social media software applications for the mental health client to use. The agreement for treatment would allow the system to track and copy the mental health client's social media postings. In addition, if permission could be obtained to track credit card purchases and other granted income purchases, such information could also be incorporated as data points in the profile. This is particularly useful to track if the mental health client makes purchases that warrant immediate response such as alcohol or firearms purchases or both. In addition, the cell phone could be preloaded with text messaging and email applications for which permission could be obtained for the system to receive copies of texts and emails sent by the mental health client with the appropriate deidentified information.

Once the requisite permissions are obtained from the mental health client and the profile is completed, the profile is associated with the cell phone, the social media accounts, and, if obtained, the text message and email accounts. The cell phone and the social media software applications used on the cell phone, and the social media accounts, text messages and emails of the mental health client are tracked over time to identify the client specific data points and triggering events specified by the profile. A log of triggering events is created. The log could record time stamps and location data from the GPS tracker, postings from social media software applications, and case logs provided by social service agencies. The identity of third parties in such logs could be removed for privacy concerns, if necessary, or are removed and only maintained by the support agency/agencies. The assigned weights are applied to each triggering event to determine whether an alert is generated. If an alert is generated, depending on the severity and scope of the alert, the mental health client's support or intervention personnel could be contacted or, if necessary, emergency services such as Mental Health Response Teams or the police.

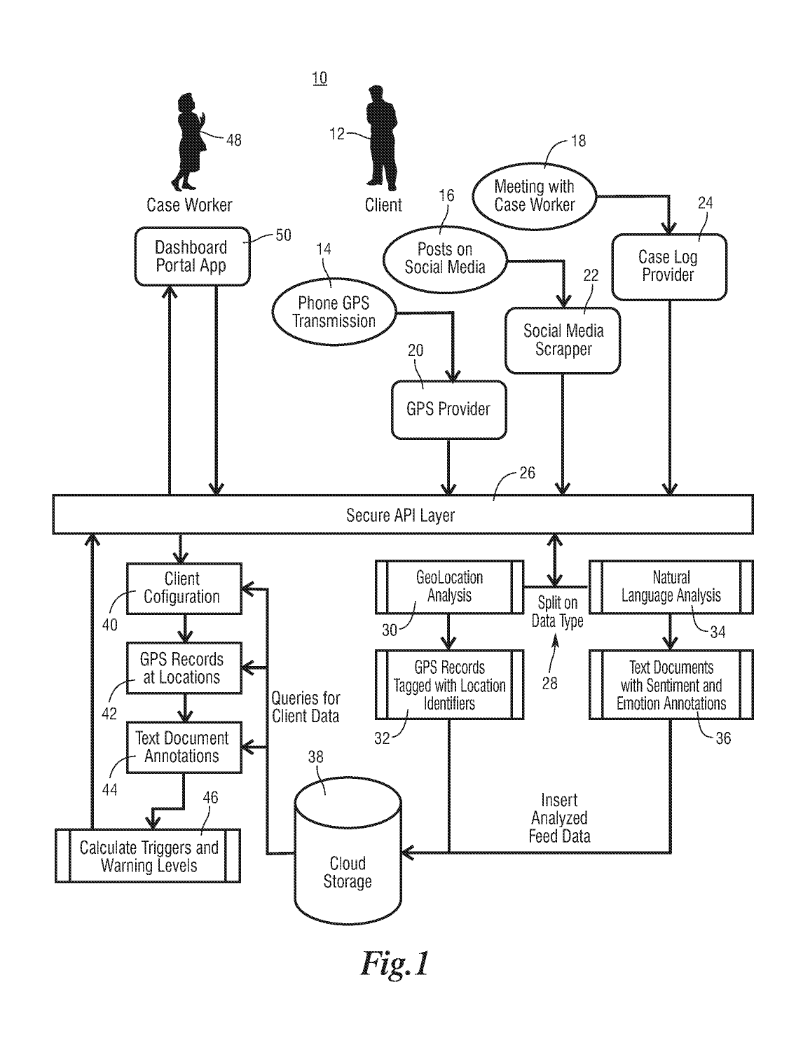

FIG. 1 shows a schematic that illustrates the implementation of the monitoring system 10 described herein. The system collects a variety of feeds from mental health clients 12, processes them through the algorithm described herein, and then displays the resulting data on a dashboard 50 that has a variety of parameters customized to each mental health client 12. The mental health client's 12 cell phone GPS transmissions 14, social media posts 16, and documented content from caseworker meetings 18 are provided by a GPS Provider 20, a social media scrapper 22, and a case log provider 24 application, respectively. The case log provider 24 uses the input of the mental health client's mental and physical health service providers to maintain and provide case file information to the system. These data points are submitted through a secure Application Programming Interface (API) 26.

Individuals who are managing serious mental illness are at risk of crisis, hospitalization, or behavior that jeopardizes personal and community safety. The cell phones distributed to such persons will use software to de-identify that client using only a cell phone code and each client has a different profile that can be weighted without compromising the Health Insurance Portability and Accountability Act (HIPAA). The cell phones can be used to access live chat rooms, texting with counselors, and even one-on-one counseling sessions and other remote technology enabled forms of care, but these are not anticipated uses of the phones. The platform is focused on patterns of cell phone usage—patterns are not a form of technology that challenges HIPAA safeguards. Patterns require meaning, weighting, and fencing to establish crisis protocols for the end goal of diverting and/or managing variances that can lead to crisis. There are opportunities when the platform contributing information needs to be re-weighted whether it be sentiment data, pattern data, or if a new cell phone is needed. The platform can adjust to weighting changes and seamless adoption of new data streams.

For the cell phone GPS transmissions 14, there are multiple tracking methods available (GPS, Assisted GPS, Synthetic GPS, Cell Identification, Wi-Fi, Inertial Sensors, Barometer, Ultrasonic, Bluetooth Beacons, and Terrestrial Transmitters). The platform will rely on any one or a combination of monitoring systems to establish the mental health client's 12 patterns, which are used to “feed” the algorithm.

After flowing through the secure API 26, the data is split 28 depending upon the nature of the data. Geolocation data (GPS data) goes through a geolocation analysis process 30 where GPS records are tagged 32 with location identifiers (such as latitude and longitude coordinates). Social media and case log information goes through a Natural Language Analysis 34 and will be processed for sentiment and emotion 36. Production of annotations for sentiment and emotions are discussed later herein.

This geolocation and sentiment and emotion data is time coded and stored in secure cloud storage 38. Because each piece of geolocation and sentiment and emotion data is time coded, sequences of events can be recreated. The secure cloud storage 38 pushes stored data in the following categories: Client Configuration 40, GPS Records at Locations 42, and Text Document Annotations 44. These are pushed through a system to calculate calibrated triggers and warning levels 46. At predetermined time periods or upon query by authorized caseworkers 48, the system will run the triggers/warning levels through the secure API 26 to a dashboard 50 that will be monitored by qualified analysts.

The codebase for all processes occurring below Secure API Layer 26 use the following open source libraries: Keras, Tensorflow, Tensorflow-Hub, Vadersentiment, Nltk, Afinn, Numpy, Pandas, Geopy, SKlearn, Shapely, Humanize, Dependency_injector, SQLalchemy, Requests, Pytz, Psycopg2-binary, Astroid, Django, Django-extensions, Django-rest-auth, Djangorestframework, Isort, Lazy-object-proxy, Mccabe, Pip, Psycopg2, Pylint, Setuptools, Six, Typed-ast, Wheel, and Wrapt.

The dashboard 50 is a portal or application that serves as a response tool during continuous (24/7) monitoring. It also serves as a query tool for caseworkers to review client information as it is being monitored by analysts. The caseworker query option can be customized to a specific grouping of information that is collapsed for ease of use or expanded to the full breath of dashboard information depending on the need of the authorized caseworker for each specific client queried. Analyst and caseworker feedback will further refine information formats and parameters while also calibrating the triggers/warning levels to optimize the dashboard 50.

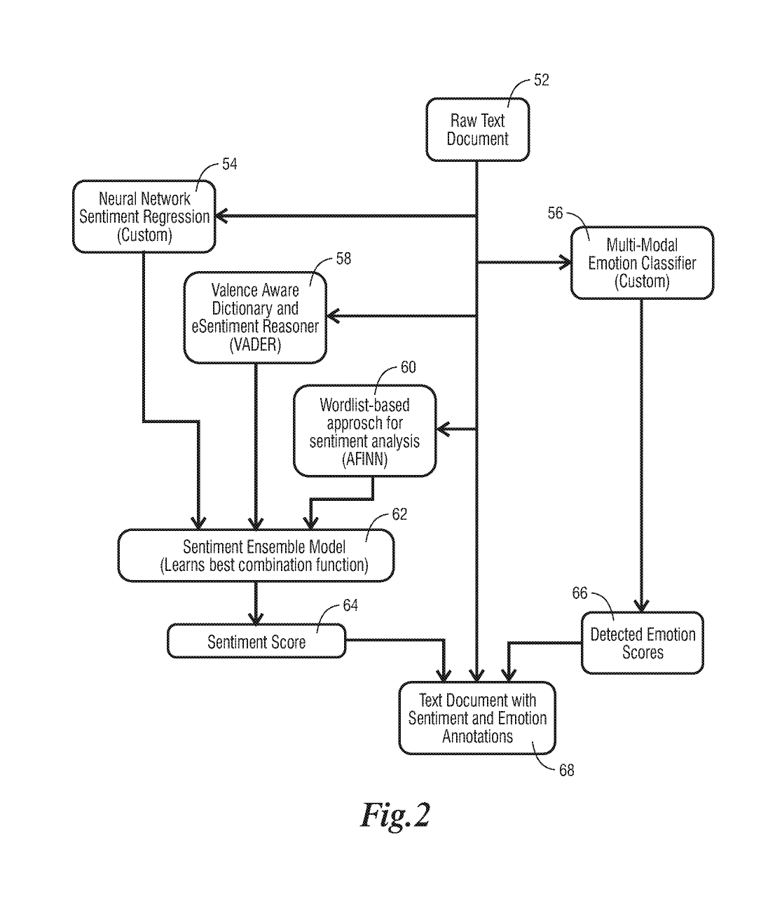

FIG. 2 shows a schematic for the natural language analysis discussed above. Raw text 52 enters the system either through client social media posts or the uploaded case notes from the service provider. Each social media post or case note uploaded is distinct with noted date and time stamps as well as origin stamps and the appropriate de-identifications. Two parallel processes are implemented on each raw text submission. On the left side of the schematic is sentiment analysis while on the right is emotion analysis.

There are three types of sentiment analysis implemented using three different algorithms. The Neural Network Sentiment Regression 54 is a custom analysis using a variety of open source code such as: Karas, Trensorflow, Numpy, Sklearn, and Numberbatch Word Embeddings. The Valence Aware Dictionary and eSentiment Reasoner (VADER) 58 is an open source sentiment analysis that compares text to an extensive list of words and phrases that have specific sentiment connotations. The wordlist-based approach for sentiment analysis (AFINN) 60 is an open source sentiment analysis that compares text to an extensive list of words and phrases that have specific sentiment connotations. The results of the use of these three sentiment analysis processing results are fed into a custom algorithm 62 which determines the best combination of the output of the three analyses using open source code such as: Karas, Trensorflow, Numpy, Sklearn, and Numberbatch Word Embeddings. A sentiment score 64 ranging from 0 (negative sentiment) to 1 (positive sentiment) is created for each raw text submission.

On the emotion analysis side, the Multi-Modal Emotion Classifier 56 again uses open source code to develop this custom algorithm, such as: Karas, Trensorflow, Numpy, Sklearn, and Numberbatch Word Embeddings. Analysis of the raw text on the following eighteen emotions is conducted: Hate, Empty, Happiness, Boredom, Disgust, Fun, Worry, Fear, Surprise, Love, Joy, Neutral, Relief, Anger, Enthusiasm, Trust, Anticipation, and Sadness. A score for each of these emotions is calculated 66. These scores range from 0 to 1 with 0.5 having a neutral rating for that emotion, 0 being indicating a lack of that emotion while 1 shows a high rate of that emotion.

The raw text with annotations for the score for sentiment detected, each of the eighteen emotion scale scores, the date and time stamps, and origin stamp are saved in the database for use in pattern analysis and alert generation 68.

Amongst the bases for determining whether an alert is warranted is the weights given to each data point. For purposes of determining the relative weight of a particular data point, the system defines each data point into categories of attributes: are they core to the treatment of the mental health client, are they affiliate attributes with third party resources that give a direct indication of the mental health client's state of mind, or are they fringe attributes from which the state of mind of the mental health client can be inferred. Each attribute is conceptualized as technology-based customer specific information that has a geo-locating pattern.

Core attributes will have unique geo-data points that are specific to community and mental health support/human services providers located in the community. Core attributes include: medication monitoring requirements and recordable behaviors such as doctor appointments to manage medication efficacy and compliance; criminal justice information as it relates to probation and parole appointments; physical health appointments such as doctor appointments for ulcers as they relate to medications with side effects that can worsen an ulcer and which requires regular doctor appointments; and support service appointments such as “Independent Living Skills”, “Group Therapy Counseling Sessions”, “Mental Health Therapist Sessions”, or “Vocational Training Courses” to build the mental health client's employable skills.

Affiliate Attributes include but are not limited to third party social media platforms such as Twitter, Facebook, and Instagram, and geotagging data. This includes sentiment and emotion analysis of posted social media content, text messages, voicemails, and emails. These are combined with the geo-content of photographs, locations, and topics made available to the platform.

Fringe Attributes will include but are not limited to digital footprint information such as spending and related geolocation pattern data, sentiment and emotion analysis monitoring, case management logs, etc. This includes monitoring credit card use for suspicious purchases. The social media postings of friends and the testimony of case managers are also included herein. Case manager logs can include a wealth of information including changes in diet, housing, and personal relationships. Changes in behavior patterns can also be noted.

Each of these attributes are weighted to contribute to the decision to create an alert. For example: Did the client miss a doctor's appointment? Did the client pick up a medication refill? Is the sentiment or emotion analysis of Facebook posts showing escalating erratic behavior or aggressive wording? Is the client making purchases outside the normal or expected pattern or geography? The process of customizing geo-points for pattern analysis can only be executed for the platform if the creators of the individual mental health client's profile have a deep expertise and knowledge of the behavioral health community services system in the municipality in which the mental health client resides. Therefore, the alert is sent to a mental health professional who is familiar with the system in which the mental health client is located. A robust behavioral health expertise is also necessary to validate the critical attributes that are important to the mental health system and an individual's variance from traditional behavior patterns.

The weighting process is a dynamic process that starts with the original profile modeling with weighting. The profile is systemically updated based on non-alert pattern shifts, input from mental health support staff, and independent evaluative weighing by mental health subject matter experts of the sentiment and emotion feeds of the social media scraping, text messages, and emails. The mental health subject matter expert updates the importance of certain locations, keeps certain locations categorized at the appropriate level, and adds new locations in order to maintain the most comprehensive pattern analysis element for the platform and alert system.

The monitoring of the platform requires analysts that are adept with monitoring content sentiment, social media feeds, and patterns that are indicative of a mental health client's stability and, more importantly, instability. The monitoring analyst is responsible for duties that requires a data science skill and an analytic skill. The data science skill set utilizes data and programming languages such as Python, R, and JavaScript to manage the data feeds in the dashboard, to manage the inputs, visualize the data, and isolate the influencing indicators. The analytic skill set is needed to empower front line professionals to employ an intelligence trade craft of both written and briefing summaries to a broad range of professionals (case managers, rehabilitation professionals, medication prescribers, peace officers, and emergency and hospital staff) who are involved in managing crisis environments and situations. The analysts will have to coordinate with other team members to effectively process alerts, system modifications, and other platform considerations to sustain an accurate situational awareness.

The collection and analysis of the data serves as a resource to field professionals both for operations reference as well as new and experienced social service employees who want to better help and manage the behavior of a mental health client. The expense of training a newly hired mental health professional, in addition to the time required to gain familiarity with the mental health client assigned to the professional, is a process the platform can improve and shorten. Having this additional analysis of the mental health client's activities will improve the learning curve necessary for new mental health support staff to understand a mental health client.

The system is primarily designed to divert crises by alerting the professional community about individual mental health client escalation events to avert a higher risk situation to the mental health client and in turn, their friends, loved ones, and the community at large.

Law enforcement is often called upon to dedicate a minimum number of officers to respond to mental health clients who are at risk to themselves or others. Officers must accompany a mental health client who is in crisis until the necessary supports are in place and a dedicated hospital accepts responsibility by admitting the person on an involuntary basis for a mandated period. The time committed to this type of call reduces the street presence of law enforcement and the cost is one that can be better utilized for other community concerns. The platform allows law enforcement to either respond earlier to the “Alert” message or they can direct a mental health client to diversion community-based services where their unstable behavior can be deescalated before the call advances to an involuntary hospital admission.

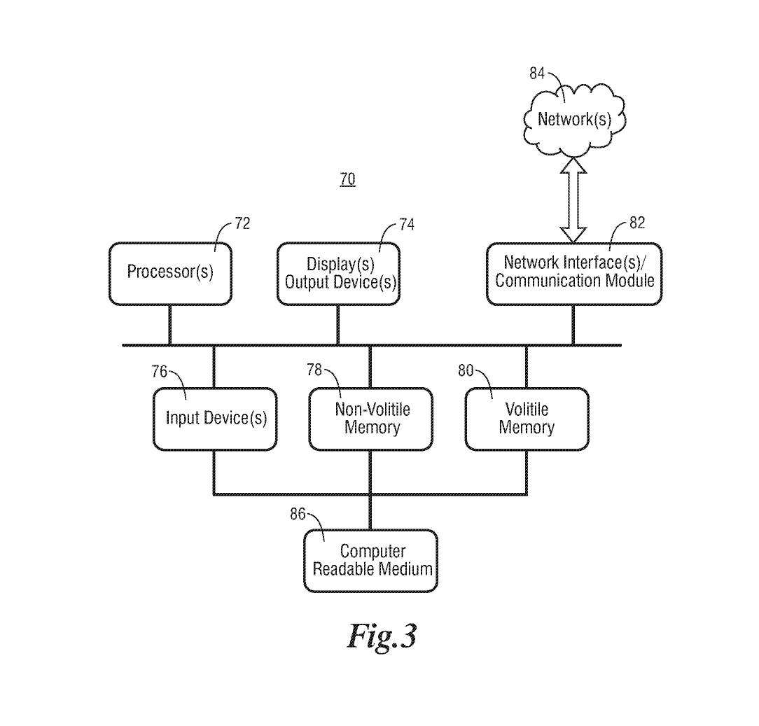

Turning now to FIG. 3, a block diagram illustrates an exemplary computing device 70, through which embodiments of the disclosure can be implemented. The computing device 70 described herein is but one example of a suitable computing device and does not suggest any limitation on the scope of any embodiments presented. Nothing illustrated or described with respect to the computing device 70 should be interpreted as being required or as creating any type of dependency with respect to any element or plurality of elements. While it is preferred that the computing device 70 is a cell phone, other embodiments may include, but need not be limited to, a desktop, laptop, server, client, tablet, smart speaker, digital assistant, or any other type of device that can compress data. in an embodiment, the computing device 70 includes at least one processor 72 and memory (non-volatile memory 78 and/or volatile memory 80). The computing device 70 can include one or more displays and/or output devices 74 such as monitors, speakers, headphones, projectors, wearable-displays, holographic displays, and/or printers, for example. Output devices 74 may further include, for example, devices that emit energy (radio, microwave, infrared, visible light, ultraviolet, x-ray and gamma ray), electronic output devices (Wi-Fi, radar, laser, etc.), audio (of any frequency), etc.

The computing device 70 may further include one or more input devices 76 which can include, by way of example, any type of mouse, keyboard, disk/media drive, memory stick/thumb-drive, memory card, pen, touch-input device, biometric scanner, voice/auditory input device, motion-detector, camera, scale, and the like, Input devices 76 may further include cameras (with or without audio recording), such as digital and/or analog cameras, still cameras, video cameras, thermal imaging cameras, infrared cameras, cameras with a charge-couple display, night-vision cameras, three-dimensional cameras, webcams, audio recorders, and the like.

The computing device 70 typically includes non-volatile memory 78 (ROM, flash memory, etc.), volatile memory 80 (RAM, etc.), or a combination thereof. A network interface 82 can facilitate communications over a network 84 via wires, via a wide area network, via a local area network, via a personal area network, via a cellular network, via a satellite network, etc. Suitable local area networks may include wired Ethernet and/or wireless technologies such as, for example, wireless fidelity (Wi-Fi). Suitable personal area networks may include wireless technologies such as, for example, IrDA, Bluetooth, Wireless USB, Z-Wave, ZigBee, and/or other near field communication protocols. Suitable personal area networks may similarly include wired computer buses such as, for example, USB and EireWire. Suitable cellular networks include, but are not limited to, technologies such as LTE, WiMAX, UMTS, CDMA, and GSM. Network interface 82 can be communicatively coupled to any device capable of transmitting and/or receiving data via one or more network(s) 84. Accordingly, the network interface hardware 82 can include a communication transceiver for sending and/or receiving any wired or wireless communication. For example, the network interface hardware 82 may include an antenna, a modern, LAN port, Wi-Fi card, WiMax card, mobile communications hardware, near-field communication hardware, satellite communication hardware and/or any wired or wireless hardware for communicating with other networks and/or devices. One or more databases may be accessed via the network(s) to remotely access data and store data.

A computer-readable medium 86 may comprise a plurality of computer readable mediums, each of which may be either a computer readable storage medium or a computer readable signal medium. A computer readable storage medium may reside, for example, within an input device 76, non-volatile memory 78, volatile memory 80, or any combination thereof. A computer readable storage medium can include tangible media that is able to store instructions associated with, or used by, a device or system. A computer readable storage medium includes, by way of example: RAM, ROM, cache, fiber optics, EPROM/Flash memory, CD/DVD/BD-ROM, hard disk drives, solid-state storage, optical or magnetic storage devices, diskettes, electrical connections having a wire, or any combination thereof. A computer readable storage medium may also include, for example, a system or device that is of a magnetic, optical, semiconductor, or electronic type. Computer readable storage media and computer readable signal media are mutually exclusive.

A computer readable signal medium can include any type of computer readable medium that is not a computer readable storage medium and may include, for example, propagated signals taking any number of forms such as optical, electromagnetic, or a combination thereof. A computer readable signal medium may include propagated data signals containing computer readable code, for example, within a carrier wave. Computer readable storage media and computer readable signal media are mutually exclusive.

The computing device 70 may include one or more network interfaces 82 to facilitate communication with one or more remote devices, which may include, for example, client and/or server devices. A network interface 82 may also be described as a communications module, as these terms may be used interchangeably.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

1. A computer-implemented method for behavioral prediction of mental health clients comprising for each mental health client:
creating a profile for each mental health client with a framework for alerts to be generated, the framework comprising:
client specific deidentified data points;
client specific weights assigned to each data point;
client specific triggering events based on each data point;
providing the mental health client with a cell phone enabled with a GPS tracker and social media software applications;
associating the profile with the cell phone and the social media software applications;
monitoring over time the cell phone and the social media software applications to identify data points and triggering events;
generating a log of triggering events
applying the assigned weights to each triggering event to determine whether or not an alert is generated;
identifying historical patterns to optimize the accuracy of future alerts.

2. The computer implemented method of claim 1 further comprising the client specific data points includes any or all of demographic, historical, clinical, diagnostic, support system information, recordable behaviors, schedules, prohibited GPS locations, prohibited GPS zones, permitted GPS locations, and diagnoses.

3. The computer implemented method of claim 1 further comprising obtaining an agreement by the mental health client to accept and use the cell phone and allow access to the data generated by it.

4. The computer implemented method of claim 1 further comprising contacting support or intervention personnel when an alert is generated.

5. The computer implemented method of claim 1 further comprising applying language analysis, sentiment analysis, and emotion analysis to data points obtained from social media software applications and case logs provided by social service agencies.

6. The computer implemented method of claim 1 further comprising recording time stamps and location data from the GPS tracker, postings from social media software applications, and case logs provided by social service agencies.

7. The computer implemented method of claim 1 further comprising deidentifying third parties from the social media software applications, case logs from social service agencies, and locations from the GPS tracker.

8. A system for behavioral prediction of mental health clients comprising:
a memory;
a processor coupled to said memory, said processor configured to:
create a profile for each mental health client with a framework for alerts to be set, said framework comprising:
client specific data points;
client specific weights assigned to each said data point;
client specific triggering events based on each said data point;
associate said profile with a cell phone enabled with a GPS tracker and social media software applications;
monitor information obtained from said cell phone;
create a log of triggering events;
apply said client specific weights to each said triggering event to determine whether or not an alert is generated;
monitor said profile and said log over time to identify historical patterns to optimize future alerts.

9. The system of claim 1 further comprising said client specific data includes any or all of demographic, historical, clinical, diagnostic, support system information, recordable behaviors, schedules, prohibited GPS locations, prohibited GPS zones, permitted GPS locations, and diagnoses.

10. The system of claim 1 further comprising obtaining an agreement by each said mental health client to accept and use said cell phone and allow access to the data generated by said cell phone.

11. The system of claim 1 further comprising contacting support or intervention personnel when an alert is generated.

12. The system of claim 1 further comprising applying language analysis, sentiment analysis, and emotion analysis to data points obtained from said social media software applications and case logs provided by social service agencies.

13. The system of claim 1 further comprising recording time stamps and location data from said GPS tracker, from posts from said social media software applications, and from case logs provided by social service providers.

14. The system of claim 1 further comprising deidentifying third parties from said social media software applications, locations from said GPS tracker, and case logs from social service providers.
